(19) United States
(12) Patent Application Publication
(10) Pub. No.: US 2024/0226362 A1
(43) Pub. Date: Jul. 11, 2024

(54) MECHANICAL SYSTEM FOR REPRODUCING SCENTS

(71) Applicants: Bryce Brichkowski, Wilton, CT (US); Eddison Nzete, Phoenix, AZ (US)

(72) Inventors: Bryce Brichkowski, Wilton, CT (US); Eddison Nzete, Phoenix, AZ (US)

(21) Appl. No.: 18/152,263

(22) Filed: Jan. 10, 2023

(51) Int. Cl. A61L 9/12; A61L 9/03

(52) U.S. Cl. CPC A61L 9/125; A61L 9/035; A61L 2209/134

(57) ABSTRACT

A mechanical system for reproducing scents includes the following: a container housing a scent; a pump for pumping the scent; a switch for selectively activating the pump; a mixer or evaporator for receiving the scent from the pump and transforming the scent into a vapor; and a media device configured to communicate with the switch for selective activation thereof.

### BACKGROUND OF THE INVENTION

The present invention relates to scent reproduction and, more particularly, to a scratch-and-sniff mechanical system that, when activated, reproduces scents of various items or environments.

As people see and hear event(s) through various media, there is no olfactory component incorporated with such occasion(s) or event(s), for better sensory perception of said occasion, event, and/or what is otherwise communicated.

Existing products/solutions do not include olfactory stimulation recording and reproduction to a media presentation as a component of communicating a moment or ongoing event, which may not be specific to the smell stimulated but communicative the associated event. Further, other inventions do not include multiple scent or odor recordings and reproductions in a media presentation of an event or immersive situation that is communicated, which is not specific to the odor or scent. Current solutions do not allow different scents or odors to be recorded and reproduced and presented alongside a media or immersive presentation as the narrative changes.

Put simply, media and immersive presentations and situations put a major focus on other senses, while neglecting input for olfactory simulation or recreation of scent from scenario or event in the future. As can be seen, there is a need for scratch and sniff mechanical systems that can store and selectively (re)produce scents of various items or environments.

### SUMMARY OF THE INVENTION

Broadly, an embodiment of the present invention provides a mechanical system that stores and reproduces scents of various items or environments. In accordance with certain aspects of the present invention, a battery, electric, phone, and/or computer-powered device is disclosed. The present invention stores and adds various scents or odors of events and scenarios to media content or a presentation to allow an individual or audience to experience the recreation of an event(s) or occasion(s) with an additional olfactory component for better perception of occasion(s) or event(s). This also provides further understanding of what is to be communicated or provides a more immersive environment to the individual or audience of scenario being recreated. Processes in accordance with the present invention are initiated just by scratching an area on an item/object.

There are no similar inventions to date. Previous media presentation prioritizes the sense of seeing, hearing, and/or vibrations, etc. The present invention integrates olfactory simulation by selective odor and scent components recording and reproduction attached to a media, environment, or specific scenario presentation (e.g., an award night recreation or bodily scent recreation). This invention allows various scents and odors to be recorded and represented alongside a media presentation or an immersive environment, to boost awareness of the moment and/or better describe what is happening to the audience viewing the media presentation or engaged in sensory perception of the scenario, media, or non-media.

As mentioned above, the present invention adds olfactory stimulation and boosts awareness of media or immersive situation or environment that is communicated by involving more senses (specifically, smell). Some event(s), presentation(s), immersive situation(s), or other forms of communication that otherwise cannot be communicated by other senses may now be communicated by olfactory stimulation using the present invention.

In one aspect of the present invention, a mechanical system for reproducing scents includes the following: a container housing a scent; a pump for pumping the scent; a switch for selectively activating the pump; a mixer or evaporator for receiving the scent from the pump and transforming the scent into a vapor; and a media device configured to communicate with the switch for selective activation thereof.

In another aspect of the present invention, the mechanical system, further including a synchronized controller configured to communicate between the media device and the switch, wherein the media device provides a display of media content, and wherein the synchronized controller selectively activates the switch based on the media content, wherein the scent is a reproduction of a past scent associated with the media content, wherein the switch activation is synchronized in time with the media content, wherein the media content is a broadcast of a past event of associated with the past scent for a portion of the time, and wherein the synchronized controller selectively activates the switch said portion of time; and further comprising a plurality of other containers, each other container having another scent wherein each other scent is fluidly selectively urged to the mixer or evaporator by the synchronized controller so that a mixture of scents are vaporized.

In yet another aspect of the present invention, a system for reproducing scents includes the following: a plurality of containers, each container housing a liquified scent; a mixer fluidly coupled to the plurality of containers; and a switch operatively associated with each container, wherein the switch is configured to, when activated, selectively urge the liquified scent to the mixer, wherein the switch is activated by impacting a surface of the switch, wherein the mixer is configured to evaporate the liquified scent through rotation; further including a heating element coupled to the mixer or an evaporator coupled to the mixer, wherein scratching the surface of the switch activates the switch.

These and other features, aspects and advantages of the present invention will become better understood with reference to the following drawings, description, and claims.

### BRIEF DESCRIPTION OF THE DRAWINGS

The following figures are included to illustrate certain aspects of the present disclosure and should not be viewed as exclusive embodiments. The subject matter disclosed is capable of considerable modifications, alterations, combinations, and equivalents in form and function, without departing from the scope of this disclosure.

FIG. 1 is a schematic diagram of an embodiment of the present invention;

FIG. 2 is a top plan view of an exemplary appliance of the embodiment of the present invention;

FIG. 3 is a block diagram of the embodiment of the present invention; and

FIG. 4 is another block diagram of the embodiment of the present invention.

### DETAILED DESCRIPTION OF THE INVENTION

The subject disclosure is described with reference to the drawings, wherein like reference numerals are used to refer to like elements throughout. In the following description, for purposes of explanation, numerous specific details are set forth to provide a thorough understanding of the present disclosure such that one skilled in the art will be enabled to make and use the present invention. It may be evident, however, that the present disclosure may be practiced without some of these specific details. Note, technical material that is known in the technical fields related to the present invention has not been described in detail so that the present invention is not unnecessarily obscured.

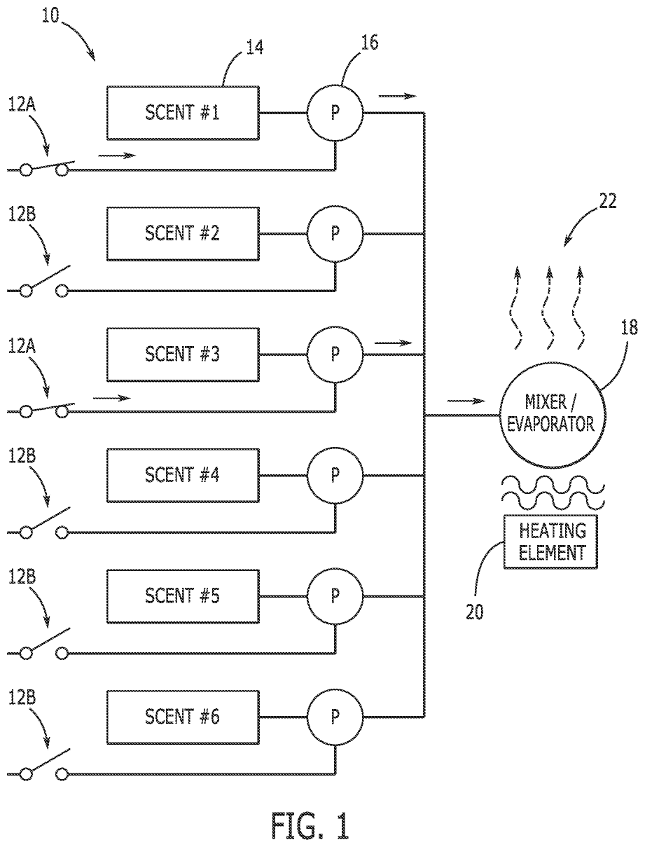

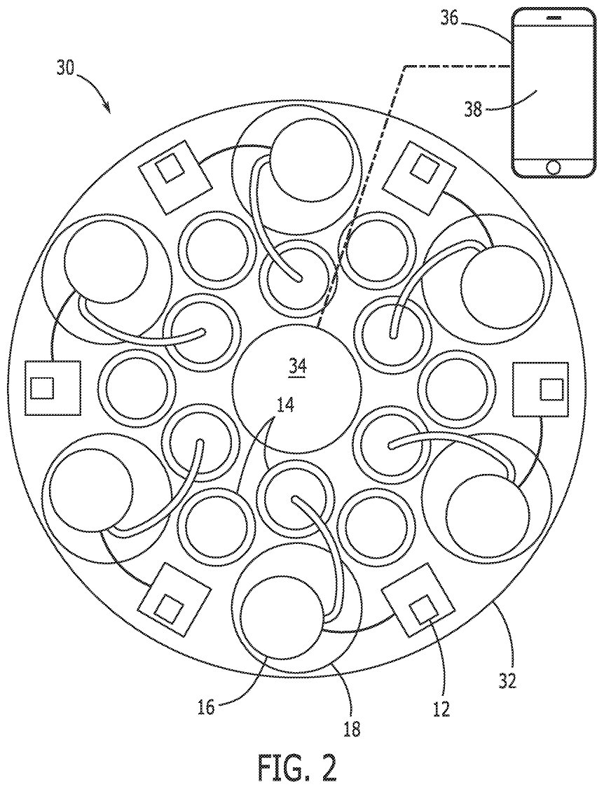

Referring now to FIGS. 1 and 2, a mechanical system 10 for producing scents of items or environments is provided. This system 10 may generally include scratch switches 12, scent vials 14, pumps 16 with controls (e.g., servo controls), a mixer and/or evaporator 18, and a heating element 20. These elements may be arranged on an appliance 30 that has a base 32, a media synchronizing controller 34 that communicates with a media device 36 with a media adapter 38. The scent vials 14 may include a scent or odor in liquid or gaseous form capable of being pumped. In certain embodiments, the scent within the scent vial 14 is a liquefied scent that is pumped to the mixer/evaporator.

As shown in FIG. 1, scratch switches 12 close circuits through scratching to supply power to a pump's servo control, which in turn provides controlled power to a pump 16. Scents or odor are obtained from a SCRATCHNSNIFF recorder A0, scents or odor are then analyzed and replicated in a SCRATCHNSNIFF replicator A1 through scent replicator data. Pump 16 is activated with power from the control and pumps scent or odor (retained, for example, in a vial 14) to a mixer 18, which can be a plain surface, or a volume inside of a motor connected container. The mixer 18 permits scent(s) and/or odor(s) to settle into or mix. The mixer 18 may be operatively associated with a heating component 20 or another form of evaporator system attached thereto. Upon being powered, as a scratch on the scratch switch 12 occurs, it will lead to heating of the mixer 18 and/or activation of an evaporation system 20, which subsequently evaporates the scent(s), odor(s) or mixture into a vapor 22. As those with skill in the art will appreciate, the mixer 18 can also function as an evaporator by continuous rotation of the receiving container of the scent/s or odor/s or mixture.

This previously described structure constitutes a basic component relationship of the present invention, colloquially known as SCRATCHNSNIFF. As seen in FIG. 2, and mentioned above, the above structure can be arranged on a base 32 that has a media synchronizing controller 34 in a middle portion thereof. When the controller 34 is connected, it can select which scratch switch 12 is to be connected for synchronization with the timing of a media presentation on a media adapter 38 (e.g., a screen on a TV, a mobile device, a projector display, etc.). Further, the media synchronizing controller 34 receives input from the media device 36, which can do so via wireless signal, such as BLUETOOTH™ or WIFI™ or wired connection, through audio output and the like. The system can function without media addon to reproduce body scent or odor stimulation.

In additional detail, and in use, the scratch switches 12 close a circuit (see 12A, for example) through scratching to supply power to the pump's servo control. The scratch switches 12 may represent multiple control pins from a servo power supply circuit. A servo circuit set (also referred to as a pump's servo control) may be used to continuously provide power. Once one or more scratch switches 12 are closed (configuration 12A shown in FIG. 1), the servo motor in the respective pump 16 is connected to the servo circuit set, turning ON, and activating the pump system 16 (e.g., a syringe/piston mechanism or non-piston mechanism) to release scent(s), odor(s), or mixture(s), depending on which and how many scratch switches 12 are scratched upon at the same time (as shown in FIG. 1). When the scratch switches 12 are open, as shown with reference number 12B, they do not activate the respective pump 16 to pump the scent 14 to the mixer/evaporator 18.

Various scent(s) and/or odor(s) are released into the mixer 18, which, as mentioned, can be a plain surface and/or the inside of a motor-connected container (this motor is connected to scratch switches 12 of each control pin, simultaneously switching ON during a scratch of the scratch switches 12 but independent of each control pin activation). A motor of the mixer 18 is not connected to a syringe mechanism, but it rotates the mixer 18 continuously whenever scratched on at the scratch switches 12—this permits scent(s) or odor(s) from the vials 14 to settle into and mix. The mixer 18 can also have a heating component 20, an evaporator, which receives and heats scent(s), odor(s), or mixture(s) until they evaporate. The evaporator 20 can also act as a receiving surface, which upon being powered as a scratch on the scratch switches 12, will lead to heating of the surface, with subsequent evaporation of the scent(s), odor(s) or mixture(s). The evaporator 20 can also function as a non-heat evaporator connected unit, releasing scent when activated.

As mentioned above, connecting media presentation to the above-described system is done using the media or scenario synchronizing controller 34, which when connected, selects which scratch switch 12, is to be activated for synchronization, with the timing of the media presentation (media synchronization does not have to be media but can be specific programmed scenario). This multiple activation of the servo motor may be achieved using a wired or wireless interphase and circuit. The media synchronizing controller 34 also receives input from media via the media device 36 which can do so by via wireless signal, such as BLUETOOTH™ or WIFI™ or wired connection, through audio output and more.

Various components that are important to the functionality of the present invention include: scratch switches 12, servo motor or motor system 16, syringe linear gear connection to servo, syringe/vial content 14, rotary container and/or heating container 18, a content release switch, servo power circuit or consistency master circuit, release content switch, audio output cable, mobile application(s), Arduino, and/or wireless/other media output interphase.

In alternate configurations of the present invention, rather than using a servo motor with a linear gear syringe system attached to servo motor, non-motor evaporators can be connected to each scratch switch. In this alternative configuration, each non-motor evaporator has a depot of a particular scent(s) and/or odor(s). The basic unit of the present invention can function with direct scratch of unit switches and sniffing scent(s) and/or odor(s) from a release point, powered either as a mechanical unit using a servo system, pump or using non-motor or another evaporator system to effect the release of scent(s) and/or odor(s).The mixer 18 can also function as an evaporator by continuous rotation of the receiving container (or mixing system unit including fan or non-motor mixer unit) of the scent(s) and/or odor(s)). A direct connection of scent(s) and/or odor(s) with individual heating or other release/evaporation system connected to the scratch switches can also achieve the intent of the present invention. Interphase can replace direct scratching of scratch switches, connected to sound, visual or other form of communication to effect release of scent(s) and/or odor(s) to include and activate olfactory stimulation to a narrative or other form of communication.

In size, the present invention may be miniature, compact, or large. The present invention may use a voltage or more less than 3 v-5 v and may use alternating or direct current. Power for the present invention may be, for example, battery, mobile power, direct current or alternating current power, laptop or computer or mobile device power, wired or wireless powered, renewable power and more. The present invention may also be powered by human motion, temperature etc.

The present invention may be used in various additional applications. For example, it may be used in memory care to evoke various smells of loved ones, locations, or occupations as media is presented or during a narrative or other form of communication. The invention can be used in virtual reality to add olfactory components to the experience. The present invention can be used in movie theaters, theme parks to communicate multiple situations or immersive moments with simultaneous olfactory stimulation, to individuals or group. The present invention can further be used to simulate various terrain or material smells in military/personal training. It can be used as a memory product release agent, used in a simulation device, in toys, in immersive experiences (e.g., movies or a theme park), as an add on or integrated product, as an animal training device, as a medical scent deliver drive, and more. For further example, the present invention could be used in the medical field as a cancer odor detection device or preventative device. In the adult film industry field, it could be used for sharing scents with viewers. For those who suffer from allergies, it could be used for detecting allergens when entering a shared space or for someone to smell a food that they cannot consume.

The present invention can be used to add olfactory stimulation to media presentation or individual scratch and sniff intent through mask, nasal prong/cannula or without. The present invention can be used to deliver inhalational medication by patients or hospital staff. The present invention can be used as a training tool (e.g., scents release representing positive re-enforcement and odor release for representing negative re-enforcement) or for scent(s) or odor exposure training. The present invention can be used in public space to create individualized environment including in airplane or other form of public and nonpublic transport. The present invention can be used with mobile and other compact devices for better media experience. The present invention can be used in theaters and large auditorium for better media experience. The present invention can also be used in simulation industry.

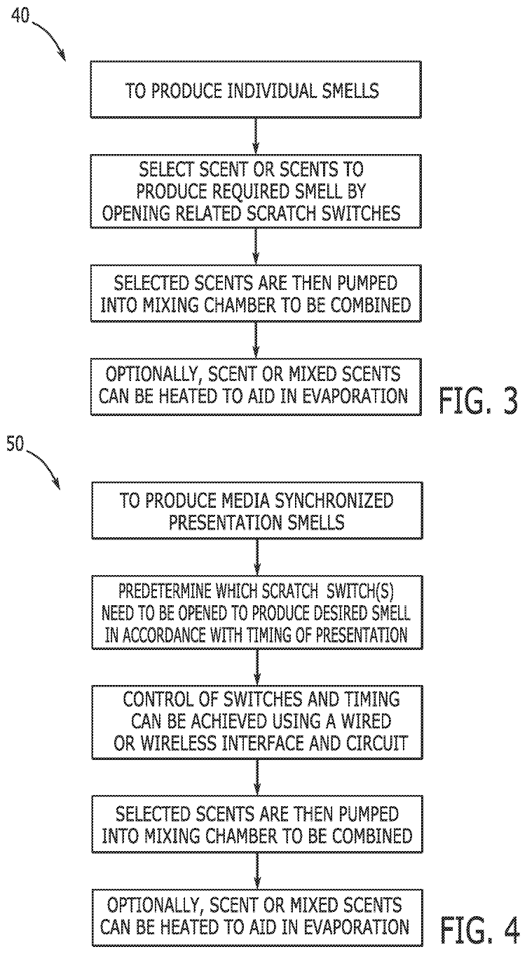

In an exemplary embodiment, the device may be used as follows. First, a syringe or vial 14 is connected to linear gear servo motor (functioning as a pump 16) using a pin or thread. Each servo motor may operatively associate with a respective syringe 14, wherein the motors are arranged in parallel, creating individual independent switches 12A/12B for each output signal connection to the servo motor. The servo system may be connected to a servo master consistency circuit and set at “Auto” using select button. The servo master consistency is powered from “in” labelled area. Each labeled scratch switch is connected to signal output with corresponding scent(s) and/or odor(s). The syringe nozzle is placed to empty into a rotary container and/or heating container. The rotary container and/or heating container is connected to a release content switch. Once the system is connected to power and one or more scratch switches 12 are activated, then a release content switch is “on”, and scent(s) and/or odor(s) are released. Block diagrams 40, 50 depicted in FIGS. 3 and 4 also generalize processes that may be employed in accordance with the present invention.

In an exemplary embodiment, the device may be made or fabricated as follows. A pump (syringe) is connected to a linear gear system of servo motor. This may be repeated with six to eight servo motors or more. All servo motors are connected in parallel, creating an independent on/off switch, between signal cable to servo motors (cable is often brownish yellow in color). This switch connection is repeated for all servo connected to linear gear mechanism. These switches are the ‘Scratch switches’. The entire servo unit is connected in parallel to servo consistency master circuit at output point, labelled Out (“s”, “−”, “+” below out sign). The servo consistency master circuit POWER INPUT “IN” (− AND + side) is connected to an external power source (3 v-5 v). The servo consistency master is set on, and at “Auto” position. The system can function without servo consistency master unit, by creating scratch switches and directly linking motor with linear gear syringe connected system, directly to 3 v-5 v and having signal point of servo or motor connected to positive power with scratch switch, controlling activation. Each syringe is filled with various scents and odors and operatively associated with a respective scratch switch, each labeled accordingly. All syringe nozzles are aligned to point to a center, preferably in a circular arrangement into a servo-controlled rotary container (with no syringe linear gear) or container (with heating/evaporating system, activated once scratch switch is on). A separate switch “content release switch” controls rotary container and heating surface/evaporator, which simultaneously comes on, whenever a scratch switch is activated. This describes a basic setup.

Media or scenario synchronicity of the system is achieved by connecting each scratch switch to a rotary system that activates a particular “servo syringe linear gear” and disconnects others using limit switches. The rotary system may receive a switch signal from wired or wireless circuit. Once the media-controlled rotary system closes the switch to a particular servo(s), scent and/or odor can be released from the unit anytime the content release switch is activated to power servo and mixer and/or evaporator.

While one or more preferred embodiments are disclosed, many other implementations will occur to one of ordinary skill in the art and are all within the scope of the invention. Each of the various embodiments described above may be combined with other described embodiments in order to provide multiple features. Furthermore, while the foregoing describes a number of separate embodiments of the apparatus and method of the present invention, what has been described herein is merely illustrative of the application of the principles of the present invention. Other arrangements, methods, modifications, and substitutions by one of ordinary skill in the art are therefore also considered to be within the scope of the present invention, which is not to be limited except by the claims that follow.

While apparatuses and methods are described in terms of “comprising,” “containing,” or “including” various components or steps, the apparatuses and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. The term “substantially” shall be interpreted to mean completely and/or nearly completely. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted. Moreover, the use of directional terms such as above, below, upper, lower, upward, downward, left, right, and the like are used in relation to the illustrative embodiments as they are depicted in the figures, the upward or upper direction being toward the top of the corresponding figure and the downward or lower direction being toward the bottom of the corresponding figure.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

What is claimed is:

1. A mechanical system for reproducing scents comprising:
a container housing a scent;
a pump for pumping the scent;
a switch for selectively activating the pump;
a mixer or evaporator for receiving the scent from the pump and transforming the scent into a vapor; and
a media device configured to communicate with the switch for selective activation thereof.

2. The mechanical system of claim 1, further comprising a synchronized controller configured to communicate between the media device and the switch, wherein the media device provides a display of media content, and wherein the synchronized controller selectively activates the switch based on the media content.

3. The mechanical system of claim 2, wherein the scent is a reproduction of a past scent associated with the media content.

4. The mechanical system of claim 3, wherein the switch activation is synchronized in time with the media content, wherein the media content is a broadcast of a past event of associated with the past scent for a portion of the time, and wherein the synchronized controller selectively activates the switch said portion of time.

5. The mechanical system of claim 4, further comprising a plurality of other containers, each other container having another scent wherein each other scent is fluidly selectively urged to the mixer or evaporator by the synchronized controller so that a mixture of scents are vaporized.

6. A system for reproducing scents comprising:
a plurality of containers, each container housing a liquified scent;
a mixer fluidly coupled to the plurality of containers; and
a switch operatively associated with each container, wherein the switch is configured to, when activated, selectively urge the liquified scent to the mixer, wherein the switch is activated by impacting a surface of the switch.

7. The system of claim 6, wherein the mixer is configured to evaporate the liquified scent through rotation.

8. The system of claim 6, further comprising a heating element coupled to the mixer.

9. The system of claim 6, further comprising an evaporator coupled to the mixer.

10. The system of claim 6, wherein scratching the surface of the switch activates the switch.
